(19) United States
(12) Patent Application Publication
(10) Pub. No.: US 2019/0316170 A1
(43) Pub. Date: Oct. 17, 2019

(54) BACTERIA DETECTION DEVICE AND BACTERIA DETECTION METHOD

(71) Applicant: SHIBASAKI INC., Chichibu-shi, Saitama (JP)

(72) Inventors: Takefumi KUDO, Isesaki-shi (JP); Yuka TAKAHASHI, Chichibu-shi (JP); Norihiro SAKAI, Osato-gun (JP); Tadashi IMAI, Kumagaya-shi (JP); Kaori ABE, Chichibu-shi (JP)

(73) Assignee: SHIBASAKI Inc., Chichibu-shi, Saitama (JP)

(21) Appl. No.: 16/314,357

(22) PCT Filed: Jun. 20, 2018
(86) PCT No.: PCT/JP2018/023421

§ 371 Date: Dec. 28, 2018

(30) Foreign Application Priority Data
Feb. 9, 2018 (JP) 2018-021886
May 14, 2018 (JP) 2018-092706

(51) Int. Cl. C12Q 1/06; B01L 3/00; G01N 21/17

(52) U.S. Cl. CPC C12Q 1/06; B01L 3/502707; B01L 3/502715; G01N 2021/174; B01L 2300/0681; B01L 2300/168; B01L 2300/0822; G01N 21/17

(57) ABSTRACT

A bacteria detection device moves a stage on which a detection chip is placed with a membrane filter fixed thereon by a first driving mechanism and a second driving mechanism in front-rear direction and in left-right direction so that an image of an upper surface of the membrane filter to which excitation light from a light emitter aligned with a imaging range by an imaging unit is taken. If bacteria to which a fluorescent label has been bound is captured on the upper surface of the membrane filter, the imaging unit can take an image of the bacteria as luminous points. A controller counts the luminous points in the image taken by the imaging unit so that the bacteria captured on the upper surface of the membrane filter can be detected.

### TECHNICAL FIELD

The present invention relates to a bacteria detection device and a bacteria detection method for detecting bacteria by causing a fluorescent label bound to the bacteria to emit fluorescence and by observing the fluorescence.

### BACKGROUND ART

According to a conventionally known bacteria detection device, bacteria captured by filtration using a membrane filter emit luminescence of ATP, and the number of the bacteria is measured by counting luminous points (for example, see Patent Document 1).

### CITATION LIST

### Patent Literature

[Patent Document 1] Japanese Unexamined Patent Application Publication No. hei6-78748

### SUMMARY OF INVENTION

### Technical Problem

Further, there has been used a bacteria detection device for detecting bacteria stained with a fluorescent reagent by irradiating the bacteria with excitation light of a predetermined wavelength range and causing a fluorescent label bound to the bacteria to emit luminescence.

As a result of intensive investigations on improvement of the bacteria detection device for detecting bacteria using such fluorescence observation, the present inventors have developed a bacteria detection device and a bacteria detection method which make it possible to detect bacteria more suitably than a conventional device.

An object of the present invention is to provide a bacteria detection device and a bacteria detection method capable of suitable bacteria detection.

### Solution to Problem

In order to achieve the above object, a bacteria detection device of an invention according to the present application includes:

a detection chip which has at least one concave portion to which a membrane filter is fixed with an upper surface of the membrane filter facing upward, the membrane filter capturing bacteria to which a fluorescent label has been bound on the upper surface;

a stage which has a placement unit on which the detection chip can be placed;

a first driving mechanism which moves the stage in one direction;

a second driving mechanism which moves the stage in a direction perpendicular to the one direction;

a light emitter which emits light to the upper surface of the membrane filter on the detection chip;

an imaging unit which takes an image of the upper surface of the membrane filter; and

a controller which counts a number of the bacteria on the membrane filter by counting luminous points in the image taken by the imaging unit.

According to the bacteria detection device configured as above, the membrane filter is fixed on the detection chip on the stage, and the first driving mechanism and the second driving mechanism move the stage in one direction and in a direction perpendicular to the one direction. As a result, it is possible to take an image while the upper surface of the membrane filter irradiated with light from the light emitter is aligned to an imaging range by the imaging unit.

If bacteria to which a fluorescent label has been bound are captured by the membrane filter fixed on the concave portion of the detection chip, the imaging unit can take an image of the bacteria as luminous points.

Thus, it is possible to suitably detect the bacteria captured on the upper surface of the membrane filter by, for example, counting the number of bacteria by counting luminous points in the image taken by the imaging unit.

Further, preferably, the bacteria detection device includes a light shielding plate which reduces reflection of light toward the concave portion, the light being emitted by the light emitter, the light shielding plate being disposed on a predetermined portion on the upper surface of the detection chip, the predetermined portion being at a side opposite to the light emitter in a state where the detection chip is placed on the stage and the concave portion is between the light emitter and the light shielding plate.

When light is emitted by the light emitter, the light shielding plate on the upper surface of the detection chip has a function to reduce reflection of light toward the concave portion. Therefore, the light shielding plate can prevent the reflected light from entering the imaging range by the imaging unit so as to make take measures against stray light. As a result, it is possible to satisfactorily take an image of the upper surface of the membrane filter fixed on the concave portion, and to perform measurement for detecting bacteria more accurately.

Further, preferably, at least the light shielding plate of the detection chip is colored in a dark color.

The light shielding plate which is colored in a dark color and does not emit fluorescence absorbs light emitted thereto. As a result, it is possible to reduce reflection of light by suitably taking measures against stray light.

If the entire detection chip is colored in a dark color, reflection of light can be also reduced at portions other than the light shielding plate, and it is possible to take even more measures against stray light.

Further, preferably, the membrane filter is fixed on a glass plate fitted in the concave portion, and, a liquid which does not emit autofluorescence is interposed between the glass plate and the membrane filter.

If the liquid which does not emit autofluorescence is interposed between the glass plate and the membrane filter, the position of the membrane filter can be stabilized due to the surface tension of the liquid.

Specifically, the membrane filter fixed on the concave portion of the detection chip keeps a position parallel to the glass plate due to the surface tension of the liquid such as water, so as to keep flatness of the upper surface of the membrane filter. Therefore, the imaging unit can take an image of the upper surface of the membrane filter satisfactorily, and accurate measurement for detecting bacteria is performed easily.

Further, preferably, the at least one concave portion of detection chip includes two concave portions.

Since two membrane filters can be fixed on the detection chip if there are two concave portions in the detection chip, it is possible to smoothly perform measurement from two samples (sample liquids).

Further, according to another invention of the present application, there is provided a bacteria detection method using the bacteria detection device according to any one of claims 1 to 5, including:

a step of capturing, by a membrane filter, bacteria to which a fluorescent label has been bound;

a step of placing a glass plate on the concave portion of the detection chip;

a step of dropping a liquid which does not emit autofluorescence on the glass plate;

a step of fixing the membrane filter on the glass plate;

a step of placing the detection chip on the stage, the membrane filter being fixed on the detection chip;

a step of emitting excitation light from the light emitter to the membrane filter;

a step of taking an image of the membrane filter with the imaging unit; and

a step of detecting the bacteria captured by the membrane filter on a basis of luminous points in the image taken by the imaging unit.

A sample liquid containing bacteria stained with a fluorescent dye (a fluorescent reagent) may be filtered by a membrane filter, so as to capture the bacteria. Alternatively, a sample liquid containing bacteria may be filtered by a membrane filter to capture the bacteria, and then the captured bacteria may be stained with a fluorescent dye (fluorescent reagent).

According to the bacteria detection method having such a configuration, if bacteria to which a fluorescent label has been bound are captured by the membrane filter fixed on the concave portion of the detection chip, it is possible to suitably detect the bacteria captured on the upper surface of the membrane filter on the basis of luminous points in the image taken by the imaging unit.

In particular, the membrane filter fixed on the concave portion of the detection chip through the glass plate keeps a position parallel to the glass plate due to the surface tension of the liquid such as water (a liquid which does not emit autofluorescence), so as to keep flatness of the upper surface of the membrane filter. Therefore, the imaging unit can take an image of the upper surface of the membrane filter satisfactorily, and accurate measurement for detecting bacteria can be performed.

Further, according to another invention of the present application, there is provided a bacteria detection method using the bacteria detection device according to any one of claims 1 to 4,

the bacteria detection device including:

a single light source which can emit predetermined excitation light as the light emitter; and

a monochromatic camera as the imaging unit,

the bacteria detection method including:

a first step of capturing, by a membrane filter, bacteria to which a fluorescent label has been bound;

a second step of placing a glass plate on the concave portion of the detection chip;

a third step of dropping a liquid which does not emit autofluorescence on the glass plate;

a fourth step of fixing the membrane filter on the glass plate;

a fifth step of placing the detection chip on the stage, the membrane filter being fixed on the detection chip;

a sixth step of emitting excitation light from the light emitter to the membrane filter;

a seventh step of taking an image of the membrane filter with the imaging unit; and

an eighth step of detecting the bacteria captured by the membrane filter on a basis of luminous points in the image taken by the imaging unit,

wherein a total number of bacteria is detected, by performing processing of the first step to the eighth step using a fluorescent label capable of binding to both viable bacteria and dead bacteria, and

wherein a number of viable bacteria or a number of dead bacteria is detected, by performing processing of the first step to the eighth step using a fluorescent label capable of binding to viable bacteria or dead bacteria.

According to the bacteria detection method using the bacteria detection device including a monochromatic camera, it is possible to measure the number of bacteria corresponding to two fluorescent labels using a single excitation light source.

Specifically, when an image of luminous points of bacteria is taken by the monochromatic camera (the imaging unit), it is possible to separately recognize the fluorescence of a fluorescent label capable of binding to both viable bacteria and dead bacteria and the fluorescence of a fluorescent label capable of binding to either viable bacteria or dead bacteria, to detect the total number of bacteria (the number of bacteria equivalent to the number of viable bacteria plus the number of dead bacteria), and to detect the number of viable bacteria or the number of dead bacteria.

As a result, it is possible to detect the number of viable bacteria on the basis of the difference between the total number of bacteria and the number of dead bacteria, to detect the number of dead bacteria on the basis of the difference between the total number of bacteria and the number of viable bacteria, and the like.

Further, it is possible to calculate the ratio of the number of dead bacteria to the total number of bacteria, the ratio of the number of viable bacteria to the total number of bacteria, and the like.

Further, according to another invention of the present application, there is provided a bacteria detection method using the bacteria detection device according to claim 5,

the bacteria detection device including:

a single light source which can emit predetermined excitation light as the light emitter; and

a monochromatic camera as the imaging unit,

the bacteria detection method including:

a first step of capturing, by a membrane filter, bacteria to which a fluorescent label has been bound;

a second step of placing a glass plate on the concave portions of the detection chip;

a third step of dropping a liquid which does not emit autofluorescence on the glass plate;

a fourth step of fixing the membrane filter on the glass plate;

a fifth step of placing the detection chip on the stage, the membrane filter being fixed on the detection chip;

a sixth step of emitting excitation light from the light emitter to the membrane filter;

a seventh step of taking an image of the membrane filter with the imaging unit; and

an eighth step of detecting the bacteria captured by the membrane filter on a basis of luminous points in the image taken by the imaging unit,

wherein, after fixing a membrane filter in one of the concave portions of the detection chip by performing processing of the first step to the fourth step using a fluorescent label capable of binding to both viable bacteria and dead bacteria, while the fluorescent label capable of binding to both viable bacteria and dead bacteria has been bound to bacteria captured by the membrane filter and

after fixing a membrane filter in another of the concave portions of the detection chip by performing processing of the first step to the fourth step using a fluorescent label capable of binding to viable bacteria or dead bacteria, while the fluorescent label capable of binding to either viable bacteria or dead bacteria has been bound to bacteria captured by the membrane filter,

the detection chip is placed on the stage by performing processing of the fifth step,

wherein a total number of bacteria is detected by performing processing of the sixth step to the eighth step on the membrane filter fixed on the one concave portion, and

wherein a number of viable bacteria or a number of dead bacteria is detected by performing processing of the sixth step to the eighth step on the membrane filter fixed on the another of the concave portions.

According to the bacteria detection method using the bacteria detection device including a monochromatic camera, it is possible to measure the number of bacteria corresponding to two fluorescent labels with a single excitation light source.

Thus, as described above, it is possible to detect the number of viable bacteria on the basis of the difference between the total number of bacteria and the number of dead bacteria, to detect the number of dead bacteria on the basis of the difference between the total number of bacteria and the number of viable bacteria, and the like.

Further, it is possible to calculate the ratio of the number of dead bacteria to the total number of bacteria, the ratio of the number of viable bacteria to the total number of bacteria, and the like.

In particular, by using the detection chip having the two concave portions, it is possible to smoothly measure the number of bacteria to which a fluorescent label capable of binding to both viable bacteria and dead bacteria has been bound, and the number of bacteria to which a fluorescent label capable of binding to either viable bacteria or dead bacteria has been bound, without replacing the membrane filter and the detection chip.

### Advantageous Effects of Invention

According to the present invention, bacteria can be suitably detected.

### BRIEF DESCRIPTION OF DRAWINGS

FIG. 1A is an exploded perspective view showing a detection chip to be attached to a bacteria detection device of the present embodiment.

FIG. 1B is a perspective view showing a detection chip to be attached to the bacteria detection device of the present embodiment.

FIG. 1C is a plan view showing a detection chip to be attached to the bacteria detection device of the present embodiment.

FIG. 2 is a perspective view showing the bacteria detection device of the present embodiment.

FIG. 3 is a perspective view showing the bacteria detection device of the present embodiment.

FIG. 4 is an enlarged perspective view showing a stage portion of the bacteria detection device of the present embodiment.

FIG. 5 is a side view showing an imaging unit of the bacteria detection device of the present embodiment.

FIG. 6A is an exploded perspective view showing a modification example of the detection chip.

FIG. 6B is a perspective view showing the modification example of the detection chip.

FIG. 7A is an exploded perspective view showing a modification example of the detection chip.

FIG. 7B is a perspective view showing the modification example of the detection chip.

### DESCRIPTION OF EMBODIMENTS

Hereinafter, embodiments of a bacteria detection device and a bacteria detection method according to the present invention will be described in detail with reference to the drawings. Although various technically preferable limitations are given to the following embodiments in order to implement the present invention, the scope of the present invention is not limited to the following embodiments and examples of the drawings.

The bacteria detection device 100 of the present embodiment is a device for detecting bacteria in a sample liquid using a measurement principle including a fluorescent dyeing method and a membrane filter method in combination.

First, the detection chip 10 to be attached to the bacteria detection apparatus 100 will be described.

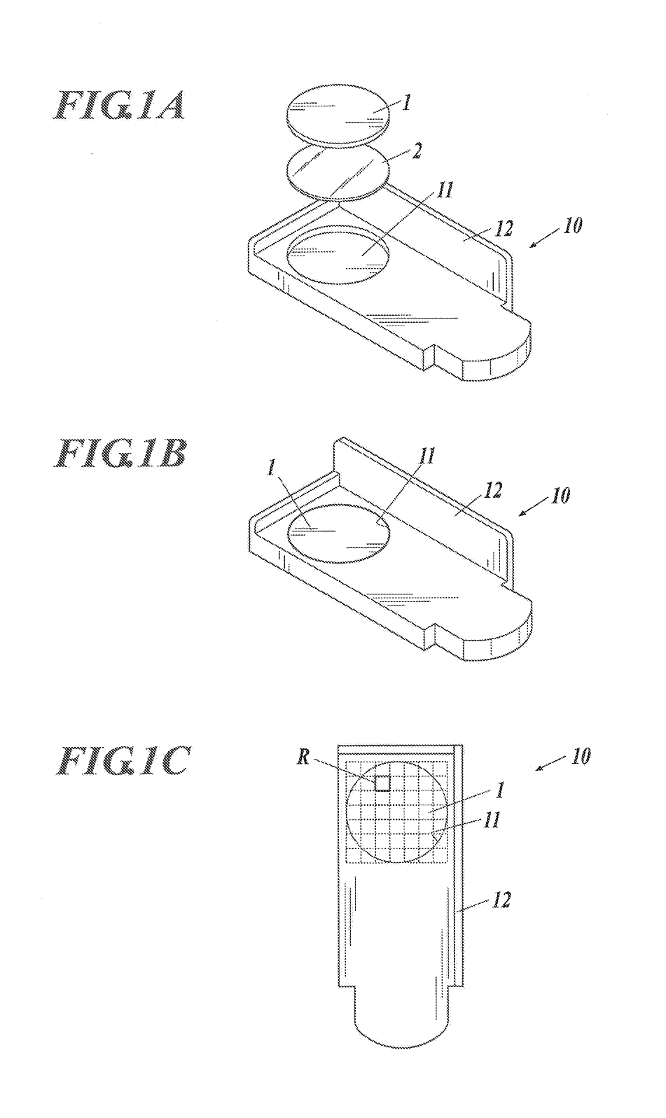

On the upper surface of the detection chip 10, as shown in FIGS. 1A and 1B, there is a concave portion 11 in which a membrane filter 1 is disposed and fixed with the upper surface thereof facing upward.

The membrane filter 1 is fixed on the concave portion 11 so as to be attached to the detection chip 10 and, on the upper surface thereof, captures bacteria to which a fluorescent label has been bound.

The sample liquid containing bacteria stained with a fluorescent dye (a fluorescent reagent) may be filtered by the membrane filter 1, so as to capture the bacteria. Alternatively, the sample liquid containing bacteria may be filtered by the membrane filter 1 to capture the bacteria, and then the captured bacteria may be stained with a fluorescent dye (a fluorescent reagent).

The membrane filter 1 attached to the concave portion 11 of the detection chip 10 is fixed on a glass plate 2 fitted in the concave portion 11, such that there is water, a liquid which does not emit autofluorescence, interposed between the glass plate 2 and the membrane filter 1.

For example, the concave portion 11 of the detection chip 10 has the glass plate 2 fitted in in advance, and the membrane filter 1 is attached to the concave portion 11 in a state where a predetermined amount of a water droplet is put on the glass plate 2.

By the water interposed between the glass plate 2 and the membrane filter 1 as described above, the position of the membrane filter 1 is stabilized in the concave portion 11 due to the surface tension of the water.

Specifically, the membrane filter 1 fixed on the concave portion 11 of the detection chip 10 keeps a position parallel to the glass plate 2 due to the surface tension of water, so as to keep flatness of the upper surface of the membrane filter 1. As a result, an imaging unit 60 described later can satisfactorily take an image (imaging of the upper surface of the membrane filter 1 on the detection chip 10), and accurate measurement for detecting bacteria can be performed easily.

If the bottom surface of the concave portion 11 is finished to be highly smooth by mirror finish treatment and the like, the glass plate 2 need not be used. For example, if the membrane filter 1 is fixed on the concave portion 11 while a water droplet is put on the bottom surface of the concave portion 11, it is possible to keep the position of the membrane filter 1 parallel to the bottom surface of the concave portion 11 due to the surface tension of the water.

The liquid interposed between the glass plate 2 and the membrane filter 1 is not limited to water, but may be glycerin, which is a liquid that does not emit autofluorescence.

Since glycerin is a liquid having less volatility than water, glycerin is preferably used when the it takes time for the measurement to detect bacteria.

Further, since glycerin is a liquid having higher viscosity than water, it is possible to bring the glass plate 2 and the membrane filter 1 into close contact with each other so as to keep the flatness of the upper surface of the membrane filter 1.

As shown in FIGS. 1A and 1B, a flange-shaped light shielding plate 12 is disposed at a predetermined position on the upper surface of the detection chip 10.

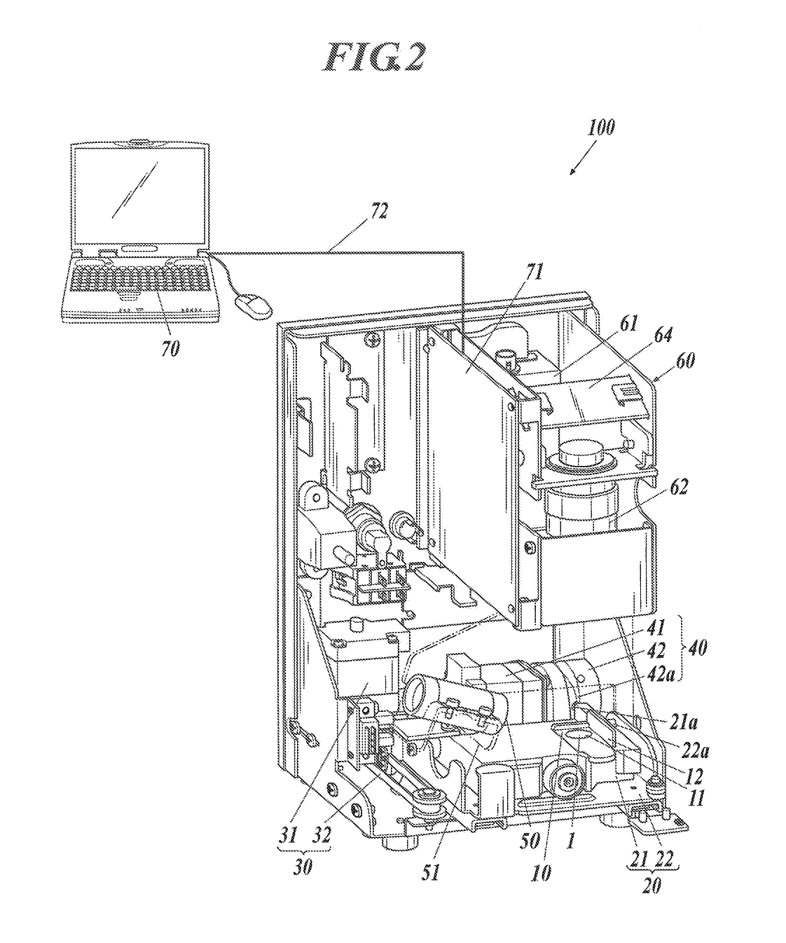

The light shielding plate 12 is disposed on the upper surface of the detection chip 10 at an opposite side of the light emitter 50 (described later) with the concave portion 11 in between, while the detection chip 10 is placed at a predetermined position of the stage 20 (described later) of the bacteria detection device 100 (see FIG. 2).

The shielding plate 12 is disposed in order to reduce the reflection of light toward the concave portion 11, when the light emitter 50 (described later) emits light toward the detection chip 10 (membrane filter 1).

Specifically, since the light shielding plate 12 is colored in a dark color and does not emit fluorescence, the light emitted onto the light shielding plate 12 is absorbed and the light reflection is reduced. As a result, it is possible to take measures against stray light by preventing reflected light from entering the imaging range by the imaging unit 60 described later.

By taking measures against stray light by reducing reflection of light using the light shielding plate 12 on the upper surface of the detection chip 10 as described above, it is possible to satisfactorily take an image (imaging of the upper surface of the membrane filter 1 on the detection chip 10) by the imaging unit 60 described later, and to perform measurement for detecting bacteria more accurately.

In the detection chip 10, at least the light shielding plate 12 needs to be colored in a dark color. In the present embodiment, however, not only the light shielding plate 12 but also the entire detection chip 10 is colored in a dark color by forming the detection chip 10 with, for example, a resin material of a dark color.

If the entire detection chip 10 is colored in a dark color, reflection of light can be also reduced at portions other than the light shielding plate 12, and it is possible to take even more measures against stray light.

Next, the bacteria detection device 100 provided with the above-described detection chip 10 will be described.

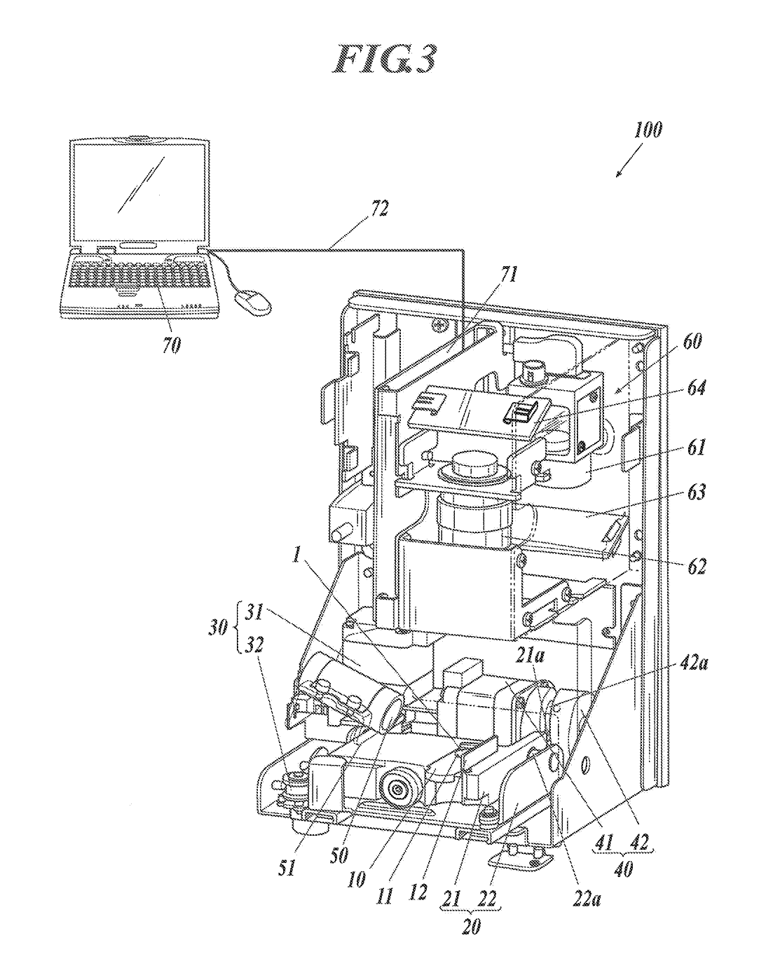

As shown in FIGS. 2 and 3, the bacteria detection device 100 of the present embodiment includes: a stage 20 to which the detection chip 10 is attached; a first driving mechanism 30 for moving the stage 20 in one direction (front-rear direction); a second driving mechanism 40 for moving the stage 20 in a direction (left-right direction) perpendicular to the one direction; a light emitter 50 for emitting light toward the detection chip 10 placed on the stage 20; an imaging unit 60 for imaging the upper surface of the membrane filter 1 on the detection chip 10 placed on the stage 20; a controller 70 for integrally controlling each unit of the device and for counting the number of bacteria on the membrane filter 1 on the basis of the image taken by the imaging unit 60; and the like.

The stage 20 includes a stage main body 21 on which the detection chip 10 is placed, and a sub stage 22 which movably supports the stage main body 21

The sub stage 22 is arranged so as to be movable back and forth along a guide (not shown) disposed in a housing of the device and extending in the front-rear direction.

The stage main body 21 is arranged movably in the left-right direction along a shaft 22a provided in the sub stage 22 and extending in the left-right direction. A placement unit to which the detection chip 10 is attached is disposed on the stage main body 21.

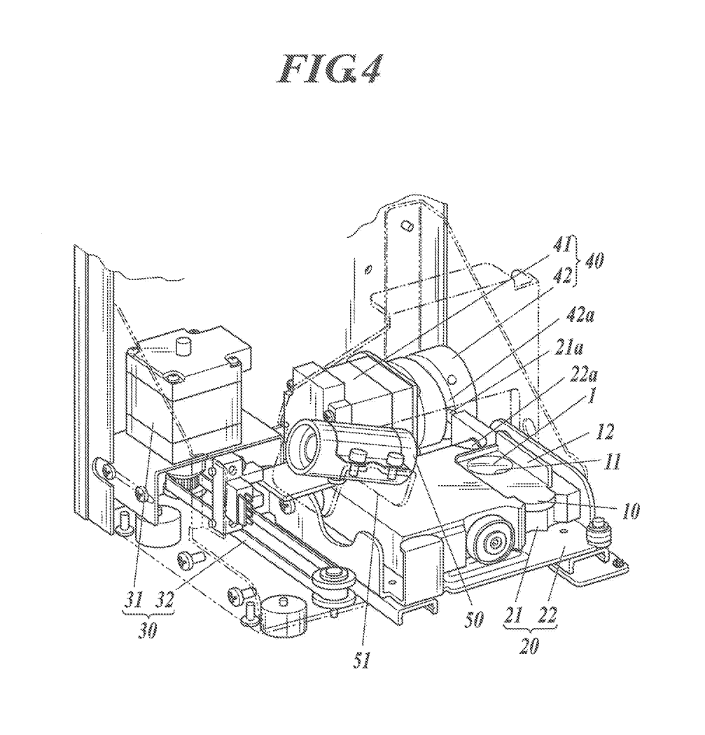

As shown in FIG. 4, the first driving mechanism 30 includes a first motor 31, a cogged belt 32 which transmits the rotational force of the first motor 31 to the sub-stage 22 of the stage 20, and the like.

The first motor 31 and the cogged belt 32 are arranged in the housing of the device, and the cogged belt 32 is connected to a part of the sub stage 22. The first motor 31 is a stepping motor.

The first driving mechanism 30 moves the sub stage 22 in the front-rear direction.

The stage main body 21 and the second driving mechanism 40 (a second motor 41, a cylindrical cam 42) on the sub stage 22 are moved in the front-rear direction together with the sub stage 22.

As shown in FIG. 4, the second driving mechanism 40 includes the second motor 41, the cylindrical cam 42 which transmits the rotational force of the second motor 41 to the stage main body 21 of the stage 20, and the like.

The second motor 41 and the cylindrical cam 42 are arranged on the sub stage 22, and a pin 21a disposed on the stage main body 21 is inserted into a helical guide groove 42a of the cylindrical cam 42. The second motor 41 is a stepping motor.

The second driving mechanism 40 moves the stage main body 21 in the left-right direction on the sub stage 22.

As shown in FIG. 4, the light emitter 50 is fixed to the housing of the device through a light source supporter 51 which supports the light emitter 50 to be arranged above the stage 20.

The light emitter 50 has a semiconductor laser (LD: Laser Diode), for example, and irradiates the upper surface of the membrane filter 1 on the detection chip 10 placed on the stage main body 21 (stage 20) with laser light as excitation light.

Specifically, the light emitter 50 emits laser light (excitation light) toward the membrane filter 1 along the left-right direction, obliquely from above.

The light emitter 50 irradiates an imaging range by the imaging unit 60 with laser light so as to emit the laser light (excitation light) toward the membrane filter 1 which is in the imaging range.

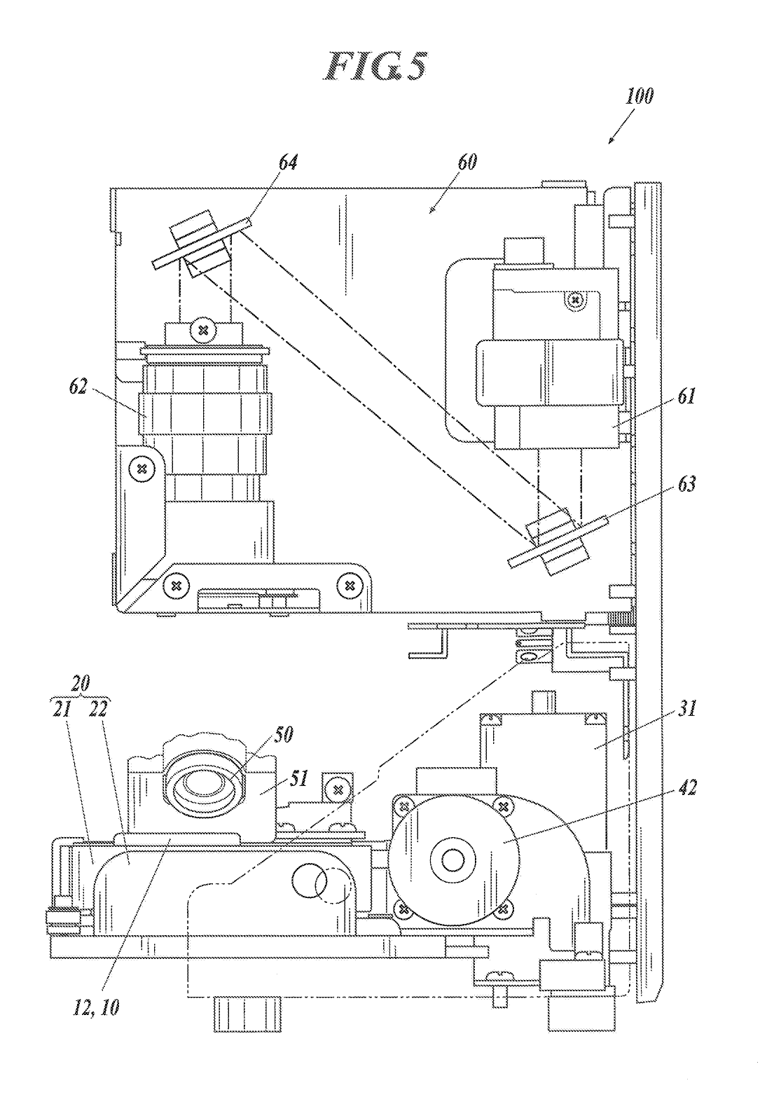

As shown in FIG. 5, the imaging unit 60 includes, for example, a CCD camera 61, a lens unit 62, mirrors 63 and 64 for folding the optical path between the CCD camera 61 and the lens unit 62, and the like.

The CCD camera 61 takes an image of the detection chip 10 placed on the stage main body 21 (the stage 20) through the lens unit 62.

Specifically, the CCD camera 61 (imaging unit 60) divides the upper surface of the membrane filter 1 on the detection chip 10 to take an image. In the present embodiment, for example, as shown in FIG. 1C, the membrane filter portion in the detection chip 10 is divided into 49 squares (7×7) to take an image.

On the upper surface of the membrane filter 1, bacteria to which a fluorescent label has been bound are captured.

The controller 70 is a personal computer such as a notebook computer connected to a control board 71 of the device through a cable 72, for example, and is provided with an operation unit such as a keyboard and a mouse, a display such as a liquid crystal display, and the like.

The controller 70 stores a program for controlling the bacteria detection device 100, and gives an operation command to the control board 71 of the device. Then, the control board 71 is configured to give the operation command to each part of the device such that the stage 20 (the stage main body 21, the sub stage 22) is moved by the operation of the first driving mechanism 30 (the first motor 31) and the second driving mechanism 40 (the second motor 41), laser light is emitted toward the detection chip 10 placed on the stage main body 21 (the stage 20) by the operation of the light emitter 50, and the like.

Further, the controller 70 is configured to give an operation command to the imaging unit 60 (the CCD camera 61), such that the imaging unit 60 can take an image of the upper surface of the membrane filter 1 on which bacteria to which the fluorescent label has been bound are captured.

In particular, the controller 70 performs processing of counting the number of bacteria on the membrane filter 1 on the basis of the image (the image of the upper surface of the membrane filter 1 on which the bacteria to which the fluorescent label has been bound are captured) taken by the imaging unit 60 (the CCD camera 61).

Specifically, the controller 70 counts the number of bacteria captured on the upper surface of the membrane filter 1 by counting the luminous points in the image taken by the imaging unit 60.

Next, processing of counting the number of bacteria in the sample liquid by the bacteria detection device 100 of the present embodiment will be described.

First, a predetermined amount of the sample liquid is filtered by the membrane filter 1.

Further, the glass plate 2 is placed on the concave portion 11 of the detection chip 10, and water, a liquid which does not emit autofluorescence, is dropped onto the glass plate 2.

Then, while a fluorescent label such as DAFT has been bound to bacteria captured on the upper surface of the membrane filter 1, the membrane filter 1 is attached to the glass plate 2 of the concave portion 11 of the detection chip 10 with water interposed therebetween, and the detection chip 10 is set on the stage main body 21 (stage 20).

Next, when the personal computer as the controller 70 is operated to start measurement of the number of bacteria by the bacteria detection device 100, the light emitter 50 emits laser light (excitation light) toward the membrane filter 1 on the detection chip 10, and the first driving mechanism 30 and the second driving mechanism 40 move the stage 20 (the stage main body 21, the sub stage 22) to a predetermined position so that the membrane filter 1 on the detection chip 10 placed on the stage 20 is aligned with an imaging area R by the imaging unit 6. Then, the imaging unit 60 (a CCD camera 61) takes an image of the upper surface of the membrane filter 1 at the predetermined position.

At this time, since measures against stray light are taken using the light shielding plate 12 on the upper surface of the detection chip 10, the imaging unit 60 (the CCD camera 61) can suitably take an image of the upper surface of the membrane filter 1.

If bacteria to which a fluorescent label has been bound are captured on the upper surface of the membrane filter 1, the imaging unit 60 (CCD camera 61) can take an image of the bacteria as luminous points. Since techniques for causing a fluorescent label to emit light using excitation light to take an image of the bacteria to which the fluorescent label has been bound as luminous points are well known, they are not described here in detail.

The imaging area R by the imaging unit 60 of the bacteria detection device 100 corresponds to, for example, one square out of the 49 squares shown in FIG. 1C. Therefore, an image of the entire region of the membrane filter 1 is taken by repeating alignment and taking of an image a plurality of times (here, 49 times).

Specifically, the stage 20 (the stage body 21) is moved in the left-right direction by the second driving mechanism 40 to align the imaging area R by the imaging unit 60 with any of the seven rows constituting the 49 squares. Then, while the first driving mechanism 30 move the stage 20 (the sub-stage 22) in the front-rear direction, images of the rows each divided into seven portions are taken.

Since the second driving mechanism 40 of the bacteria detection device 100 moves the stage 20 (the stage main body 21) through the cylindrical cam 42, it is suitable for movement over a relatively short distance, such as alignment of the imaging area R by the imaging unit 60 with one of the seven rows in the 49 squares arranged in the left-right direction.

Meanwhile, since the first driving mechanism 30 of the bacteria detection device 100 moves the stage 20 (the sub stage 22) through the cogged belt 32, it is suitable for movement over a relatively long distance, such as alignment of the imaging area R by the imaging unit 60 with each portion along the row.

Next, the controller 70 performs image processing of removing overlapping portions of the multiple images taken by the imaging unit 60 and processing of counting luminous points in the images of the respective squares (the 49 squares), so as to count the number of bacteria captured on the upper surface of the membrane filter 1.

By measuring the number of bacteria captured on the upper surface of the membrane filter 1 as described above, it is possible to detect the number of bacteria per 1 cc of the sample liquid after filtration by a filter, for example.

As described above, using the bacteria detection device 100 (bacteria detection method) of the present embodiment, bacteria captured on the upper surface of the membrane filter 1 can be suitably detected.

In particular, by taking measures against stray light using the light shielding plate 12 of the detection chip 10 of the bacteria detection device 100 and by securing the flatness of the upper surface of the membrane filter 1 fixed on the concave portion 11 of the detection chip 10 due to the surface tension of a liquid such as water (a liquid that does not emit autofluorescence), it is possible to suitably take an image of the upper surface of the membrane filter 1 attached to the detection chip 10. As a result, it is possible to suitably detect the bacteria captured on the upper surface of the membrane filter 1 on the basis of the image.

The bacteria detection device 100 can be used not only for accurately counting the number of bacteria captured on the upper surface of the membrane filter 1 (bacteria contained in the sample liquid), but also for determining whether the number of the bacteria is equal to or larger than the threshold value, or less than the threshold value. The bacteria detection processing can be finished in a shorter time for the processing of determining whether or not the number of bacteria is equal to or larger than the threshold value, than for the processing of counting the number of all the bacteria.

In the above embodiment, the imaging unit 60 of the bacteria detection device 100 takes images of the membrane filter portion of the detection chip 10 divided into 49 squares (7×7), however, the present invention is limited thereto. For example, the number of division (the number of squares) in taking images may be any arbitrary number, according to the imaging magnification of the imaging unit 60 or the like.

Next, as another embodiment regarding the measurement of the number of bacteria using the bacteria detection device 100 of the present embodiment, measurement of the number of bacteria using a single excitation light source and two fluorescent labels (fluorescent reagents) will be described.

Only the parts different from the above embodiment will be described, and descriptions will be omitted regarding the same configurations as in the above embodiment.

Conventionally, a method including the following has been adopted in some cases: performing detection processing of the total number of bacteria (the number of bacteria equivalent to the number of viable bacteria plus the number of dead bacteria) using a fluorescent label capable of binding to both viable bacteria and dead bacteria; performing detection processing of the number of dead bacteria using a fluorescent label capable of binding to dead bacteria; and detecting the number of viable bacteria on the basis of the difference between the total number of bacteria and the number of dead bacteria.

However, the conventional technique requires two excitation light sources: the first excitation light source capable of emitting excitation light corresponding to the fluorescent label capable of binding to both viable bacteria and dead bacteria; and the second excitation light source capable of emitting excitation light corresponding to the fluorescent label capable of binding to dead bacteria. As a result of intensive studies, the present inventors have developed a technique which enables measurement of the number of bacteria corresponding to two fluorescent labels using a single excitation light source.

The bacteria detection device 100 used for the measurement of the number of bacteria is provided with a single excitation light source which is capable of emitting predetermined excitation light as a light emitter 50 and a monochromatic camera as the imaging unit 60.

The light emitter 50 capable of emitting excitation light of a predetermined wavelength has, for example, a semiconductor laser (LD: Laser Diode), which is an excitation light source capable of emitting a laser beam (excitation light) having a wavelength of 405 nm.

The CCD camera 61 in the imaging unit 60 is a monochromatic CCD camera. An excitation cut filter which transmits light having a wavelength of 450 nm or more is arranged in the lens unit 62 of the imaging unit 60, so as not to transmit light having a wavelength of 405 nm.

Examples of the two fluorescent labels (fluorescent reagents) used for the measurement of the number of bacteria may be DAPI (4′,6-diamidino-2-phenylindole) as a fluorescent label capable of binding to both viable bacteria and dead bacteria, and AO (acridine orange) as a fluorescent label capable of binding to dead bacteria.

In the present embodiment, DAPI and AO were used as the two fluorescent labels (fluorescent reagents).

The present inventors found that, when the light emitter 50 emits excitation light of a predetermined wavelength toward the membrane filter 1 on which bacteria to which the fluorescent labels have been bound are captured and when the imaging unit 60 including a monochromatic CCD camera (CCD camera 61) takes an image of the luminous points of bacteria, fluorescence (luminous points) of DAPI and fluorescence (luminous points) of AO can be each suitably recognized. Specifically, when a laser beam having a wavelength of 405 nm is emitted, the fluorescence intensity from DAPI and the fluorescence intensity from AO are different, and it is possible to suitably recognize the corresponding luminous points.

That is, the present inventors found that it is possible to measure the number of bacteria corresponding to two fluorescent labels with a single excitation light source if a monochromatic CCD camera is used.

Here, the total number of bacteria (the number of bacteria equivalent to the number of viable bacteria plus the number of dead bacteria) can be detected by measuring the number of bacteria to which DAPI has been bound (the number of luminous points), and the number of dead bacteria can be detected by measuring the number of bacteria to which AO has been bound (the number of luminous points).

Then, as described later, the number of viable bacteria can be obtained by calculating the difference between the number of bacteria to which DAPI has been bound (the number of bacteria (the total number of bacteria) equivalent to the number of viable bacteria plus the number of dead bacteria) and the number of bacteria to which AO has been bound (the number of dead bacteria).

The present inventors confirmed that there is a correlation between the number of viable bacteria determined by the measurement of the number of bacteria as above including calculating the difference between the total number of bacteria and the number of dead bacteria, and the number of viable bacteria determined by a conventionally known culture method (a method including counting the number of colonies formed after application of a sample to an agar medium). Therefore, the present inventors determined that it is effective to measure the number of bacteria using a monochromatic CCD camera, along with a single excitation light source and two fluorescent labels.

Next, the measurement of the number of bacteria using a single excitation light source and two fluorescent labels will be specifically described.

First, while bacteria to which DAPI has been bound are captured on the upper surface of the membrane filter 1, the membrane filter 1 is attached to the glass plate 2 of the concave portion 11 of the detection chip 10 with a liquid such as water (a liquid which does not emit autofluorescence) interposed therebetween, and the detection chip 10 is set on the stage main body 21 (the stage 20).

Next, when the personal computer as the controller 70 is operated to start measurement of the number of bacteria by the bacteria detection device 100, the light emitter 50 emits excitation light of a predetermined wavelength toward the membrane filter 1 on the detection chip 10, and the first driving mechanism 30 and the second driving mechanism 40 move the stage 20 (the stage main body 21, the sub stage 22) to a predetermined position so that the membrane filter 1 on the detection chip 10 placed on the stage 20 is aligned with an imaging area R by the imaging unit 6. The imaging unit 60 (a CCD camera 61) takes an image of the upper surface of the membrane filter 1 at the predetermined position.

If bacteria to which DAPI has been bound are captured on the upper surface of the membrane filter 1, the imaging unit 60 (the CCD camera 61) can take an image of the bacteria as luminous points.

Next, the controller 70 performs processing of counting luminous points in the images, so as to count the number of bacteria captured on the upper surface of the membrane filter 1.

By measuring the number of bacteria captured on the upper surface of the membrane filter 1 as described above and to which DAPI has been bound, it is possible to detect the total number of bacteria (the number of bacteria equivalent to the number of viable bacteria plus the number of dead bacteria) per 1 cc of the sample liquid after filtration by a filter, for example.

This measurement of the number of bacteria to which DAPI has been bound is the first step.

After detecting the total number of bacteria, the detection chip 10 is removed from the stage main body 21 (the stage 20).

Next, while bacteria to which AO has been bound are captured on the upper surface of the membrane filter 1, the membrane filter 1 is attached to the glass plate 2 of the concave portion 11 of the detection chip 10 with a liquid such as water (a liquid which does not emit autofluorescence) interposed therebetween, and the detection chip 10 is set on the stage main body 21 (the stage 20).

Next, when the personal computer as the controller 70 is operated to start measurement of the number of bacteria by the bacteria detection device 100, the light emitter 50 emits excitation light of a predetermined wavelength toward the membrane filter 1 on the detection chip 10, and the first driving mechanism 30 and the second driving mechanism 40 move the stage 20 (the stage main body 21, the sub stage 22) to a predetermined position so that the membrane filter 1 on the detection chip 10 placed on the stage 20 is aligned with an imaging area R by the imaging unit 6. The imaging unit 60 (the CCD camera 61) thereby takes an image of the upper surface of the membrane filter 1 at the predetermined position.

If bacteria to which AO has been bound are captured on the upper surface of the membrane filter 1, the imaging unit 60 (CCD camera 61) can take an image of the bacteria as luminous points.

Next, the controller 70 performs processing of counting luminous points in the images, so as to count the number of bacteria captured on the upper surface of the membrane filter 1.

By measuring the number of bacteria captured on the upper surface of the membrane filter 1 as described above and to which AO has been bound, it is possible to detect the number of dead bacteria per 1 cc of the sample liquid after filtration by a filter, for example.

This measurement of the number of bacteria to which AO has been bound is the second step.

Subsequently, the controller 70 can calculate the number of viable bacteria by calculating the difference between the total number of bacteria detected in the first step and the number of dead bacteria detected in the second step

The detected total number of bacteria, the detected number of dead bacteria, and the calculated number of viable bacteria are displayed on the display.

The controller 70 may execute the processing of calculating not only the number of viable bacteria based on the difference between the total number of bacteria and the number of dead bacteria, but also the ratio of the number of dead bacteria to the total number of bacteria.

As described above, according to the bacteria detection device 100 (the bacteria detection method) of the present embodiment, the measurement of the number of bacteria can be suitably performed using a single excitation light source and two fluorescent labels.

Then, it is possible to calculate the number of viable bacteria on the basis of the difference between the number of total bacteria and the number of dead bacteria, the ratio of the number of dead bacteria to the total number of bacteria, and the like.

It should be noted that the present invention is not limited to the above embodiments.

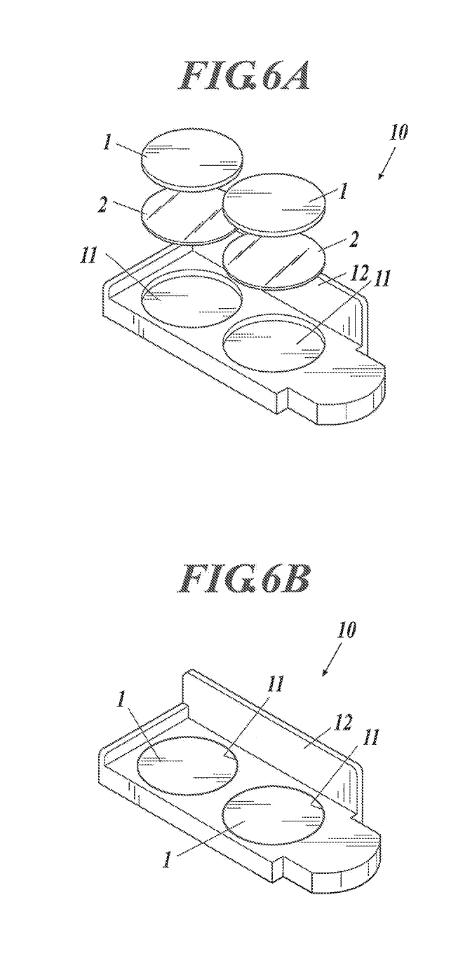

For example, as shown in FIGS. 6A and GB, the detection chip 10 having two concave portions 11 can be used as the detection chip 10 attached to the bacteria detection device 100 of the present embodiment.

The two concave portions 11 of the detection chip 10 shown in FIGS. 6A and 6B are arranged along the front-rear direction in a state where the detection chip 10 is set on the stage main body 21 (the stage 20).

If there are two concave portions 11 in the detection chip 10, the membrane filter 1 is fixed in one concave portion 11 while DAPI has been bound to bacteria captured on the upper surface of the membrane filter 1. Further, the membrane filter 1 is fixed in the other concave portion 11 while AO has been bound to bacteria captured on the upper surface of the membrane filter 1.

Measurement of the number of bacteria using the detection chip 10 having two concave portions 11 as described above, as well as using one light source for excitation and two fluorescent labels (fluorescent reagents), will be described.

First, while DAPI has been bound to bacteria captured on the upper surface of the membrane filter 1, the membrane filter 1 is fixed on the glass plate 2 of one concave portion 11 of the detection chip 10 with a liquid such as water (a liquid which does not emit autofluorescence) interposed therebetween.

Further, while AO has been bound to bacteria captured on the upper surface of the membrane filter 1, the membrane filter 1 is fixed on the glass plate 2 of one concave portion 11 of the detection chip 10 with a liquid such as water (a liquid which does not emit autofluorescence) interposed therebetween.

Then, the detection chip 10 with the membrane filters 1 each attached to the two concave portions is set on the stage main body 21 (the stage 20).

Next, when the personal computer as the controller 70 is operated to start measurement of the number of bacteria by the bacteria detection device 100, the light emitter 50 emits excitation light of a predetermined wavelength toward the membrane filter 1 on the detection chip 10, and the first driving mechanism 30 and the second driving mechanism 40 move the stage 20 (the stage main body 21, the sub stage 22) so that the membrane filter 1 on the detection chip 10 placed on the stage 20 is aligned with an imaging area R by the imaging unit 6. The imaging unit 60 (the CCD camera 61) thereby takes an image of the upper surface of the membrane filter 1 at a predetermined position. The movable range of the sub stage 22 in the front-rear direction by the first driving mechanism 30 and the movable range of the stage main body 21 in the left-right direction by the second driving mechanism 40 are adjusted, so that the membrane filter 1 fixed in each the two concave portions 11 can be aligned with the imaging area P by the imaging unit 60.

If bacteria to which DAPI has been bound are captured on the upper surface of the membrane filter 1 fixed on the one concave portion 11, the imaging unit 60 (the CCD camera 61) can take an image of the bacteria as luminous points. Similarly, if bacteria to which AO has been bound are captured on the upper surface of the membrane filter 1 fixed on the other concave portion 11, the imaging unit 60 (the CCD camera 61) can take an image of the bacteria as luminous points.

Next, the controller 70 performs processing of counting luminous points in the images taken by the imaging unit 60, so as to count the number of bacteria captured on the upper surface of the membrane filter 1.

By measuring the number of bacteria captured on the upper surface of the membrane filter 1 fixed on one concave portion as described above and to which DAPI has been bound, it is possible to detect the total number of bacteria (the number of bacteria equivalent to the number of viable bacteria plus the number of dead bacteria) per 1 cc of the sample liquid after filtration by a filter, for example.

Further, by measuring the number of bacteria captured on the upper surface of the membrane filter 1 fixed on the other concave portion and to which AO has been bound, it is possible to detect the number of dead bacteria per 1 cc of the sample liquid after filtration by a filter, for example.

Subsequently, the controller 70 calculates the number of viable bacteria by calculating the difference between the total number of bacteria and the number of dead bacteria.

The detected total number of bacteria, the detected number of dead bacteria, and the calculated number of viable bacteria are displayed on the display.

The controller 70 may execute the processing of calculating not only the number of viable bacteria based on the difference between the total number of bacteria and the number of dead bacteria, but also the ratio of the number of dead bacteria to the total number of bacteria.

As described above, according to the bacteria detection device 100 (the bacteria detection method) of the present embodiment, the measurement of the number of bacteria can be suitably performed using a single excitation light source and two fluorescent labels.

Then, it is possible to calculate the number of viable bacteria on the basis of the difference between the number of total bacteria and the number of dead bacteria, the ratio of the number of dead bacteria to the total number of bacteria, and the like.

In particular, by using the detection chip 10 having two concave portions 11, it is possible to smoothly measure the number of bacteria to which DAPI has been bound and the number of bacteria to which AO has been bound, without replacing the membrane filter 1 and the detection chip 10.

Although the two concave portions 11 of the detection chip 10 shown in FIGS. 6A and GB are arranged along the front-rear direction in a state where the detection chip 10 is set on the stage body 21 (stage 20), the invention is not limited to the above embodiment.

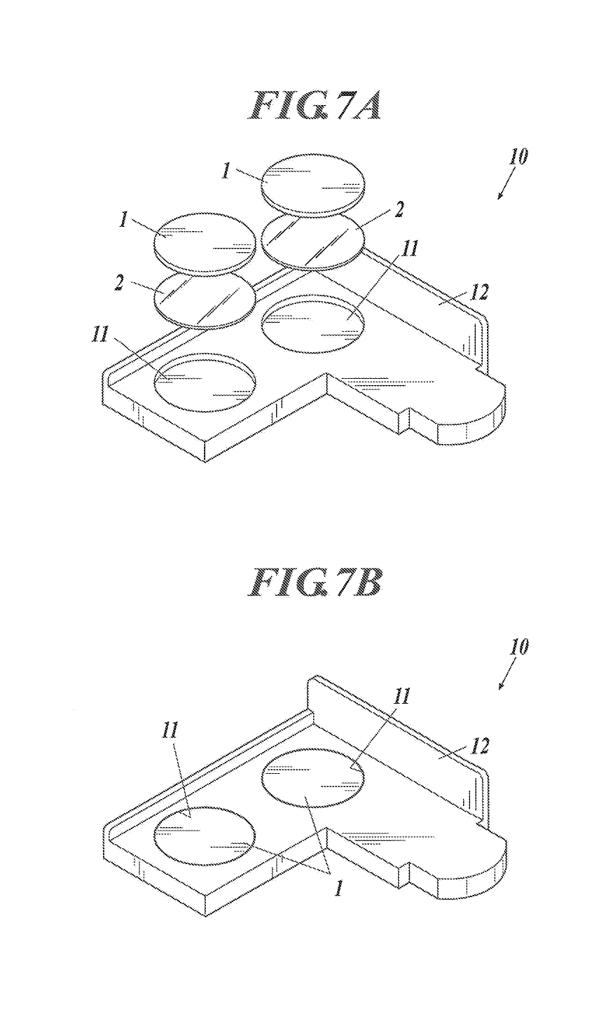

For example, as shown in FIGS. 7A and 7B, the detection chip 10 may be provided with two concave portions 11 arranged along the left-right direction in a state where the detection chip 10 is set on the stage body 21 (the stage 20).

In this case, the placement unit of the detection chip 10 in the stage main body 21 is formed in a shape corresponding to the detection chip 10 shown in FIGS. 7A and 7B.

The movable range of the sub stage 22 in the front-rear direction by the first driving mechanism 30 and the movable range of the stage main body 21 in the left-right direction by the second driving mechanism 40 are adjusted, such that the membrane filter 1 fixed in each the two concave portions 11 can be aligned with the imaging area R by the imaging unit 60.

Even with such an arrangement, by using the detection chip 10 provided with the two concave portions 11, it is possible to smoothly measure the number of bacteria to which DAPI has been bound and the number of bacteria to which AO has been bound, without replacing the membrane filter 1 and the detection chip 10.

In the above embodiment, the processing of calculating the number of viable bacteria based on the difference between the total number of bacteria and the number of dead bacteria (processing of calculating the ratio of the number of dead bacteria to the total number of bacteria) is described, however, the present invention is not limited thereto. For example, the processing of calculating the number of dead bacteria based on the difference between the total number of bacteria and the number of viable bacteria (the processing of calculating the ratio of the number of viable bacteria to the total number of bacteria) may be executed using a fluorescent label capable of binding to both viable bacteria and dead bacteria and a fluorescent label capable of binding to viable bacteria.

Further, in the above-described embodiments, the membrane filters 1 are fixed on the two concave portions 11 in the detection chip 10 while different fluorescent labels from each other have been bound to bacteria captured on the upper surface of the respective membrane filters 1, but the present invention is not limited thereto. For example, measurement of the two samples (the sample liquids) may be performed by fixing the membrane filters 1 to the two concave portions 11 in the detection chip 10 while the same fluorescent labels as each other have been bound to bacteria captured on the upper surface of the respective membrane filters 1.

In addition, it is a matter of course that specific detailed structures and the like can be changed as appropriate.

### INDUSTRIAL APPLICABILITY

The present invention is applicable to a bacteria detection device a bacteria detection method for detecting bacteria by causing a fluorescent label bound to bacteria to emit fluorescence and by observing the fluorescence.

### REFERENCE SIGNS LIST

1 Membrane Filter
2 Glass Plate
10 Detection Chip
11 Concave Portion
12 Light Shielding Plate
20 Stage
21 Stage Main Body
21a Pin
22 Sub stage
22a Shaft
30 First Driving Mechanism
31 First Motor
32 Cogged Belt
40 Second Driving Mechanism
41 Second Motor
42 Cylindrical Cam
42a Guide Groove
50 Light Emitter
51 Light Source Supporter
60 Imaging Unit
61 CCD Camera
62 Lens Unit
63, 64 Mirror
70 Controller
71 Control Substrate
73 Cable
100 Bacteria Detection Device
R Imaging Area

1. A bacteria detection device comprising:
a detection chip which has at least one concave portion to which a membrane filter is fixed with an upper surface of the membrane filter facing upward, the membrane filter capturing bacteria to which a fluorescent label has been bound on the upper surface;
a stage which has a placement unit on which the detection chip can be placed;
a first driving mechanism which moves the stage in one direction;
a second driving mechanism which moves the stage in a direction perpendicular to the one direction;
a light emitter which emits light to the upper surface of the membrane filter on the detection chip;
an imaging unit which takes an image of the upper surface of the membrane filter; and
a controller which counts a number of the bacteria on the membrane filter by counting luminous points in the image taken by the imaging unit.

2. The bacteria detection device according to claim 1, comprising a light shielding plate which reduces reflection of light toward the concave portion, the light being emitted by the light emitter, the light shielding plate being disposed on a predetermined portion on the upper surface of the detection chip, the predetermined portion being at a side opposite to the light emitter in a state where the detection chip is placed on the stage and the concave portion is between the light emitter and the light shielding plate.

3. The bacteria detection device according to claim 2, wherein at least the light shielding plate of the detection chip is colored in a dark color.

4. The bacteria detection device according to claim 1,
wherein the membrane filter is fixed on a glass plate fitted in the concave portion, and
wherein a liquid which does not emit autofluorescence is interposed between the glass plate and the membrane filter.

5. The bacteria detection device according to claim 1, wherein the at least one concave portion of detection chip includes two concave portions.

6. A bacteria detection method using the bacteria detection device according to claim 1, comprising:
a step of capturing, by a membrane filter, bacteria to which a fluorescent label has been bound;
a step of placing a glass plate on the concave portion of the detection chip;
a step of dropping a liquid which does not emit autofluorescence on the glass plate;
a step of fixing the membrane filter on the glass plate;
a step of placing the detection chip on the stage, the membrane filter being fixed on the detection chip;
a step of emitting excitation light from the light emitter to the membrane filter;
a step of taking an image of the membrane filter with the imaging unit; and
a step of detecting the bacteria captured by the membrane filter on a basis of luminous points in the image taken by the imaging unit.

7. A bacteria detection method using the bacteria detection device according to claim 1,
the bacteria detection device comprising:
a single light source which can emit predetermined excitation light as the light emitter; and
a monochromatic camera as the imaging unit,
the bacteria detection method comprising:
a first step of capturing, by a membrane filter, bacteria to which a fluorescent label has been bound;
a second step of placing a glass plate on the concave portion of the detection chip;
a third step of dropping a liquid which does not emit autofluorescence on the glass plate;
a fourth step of fixing the membrane filter on the glass plate;
a fifth step of placing the detection chip on the stage, the membrane filter being fixed on the detection chip;
a sixth step of emitting excitation light from the light emitter to the membrane filter;
a seventh step of taking an image of the membrane filter with the imaging unit; and
an eighth step of detecting the bacteria captured by the membrane filter on a basis of luminous points in the image taken by the imaging unit,
wherein a total number of bacteria is detected, by performing processing of the first step to the eighth step using a fluorescent label capable of binding to both viable bacteria and dead bacteria, and
wherein a number of viable bacteria or a number of dead bacteria is detected, by performing processing of the first step to the eighth step using a fluorescent label capable of binding to viable bacteria or dead bacteria.

8. A bacteria detection method using the bacteria detection device according to claim 5,
the bacteria detection device comprising:
a single light source which can emit predetermined excitation light as the light emitter; and
a monochromatic camera as the imaging unit,
the bacteria detection method comprising:
a first step of capturing, by a membrane filter, bacteria to which a fluorescent label has been bound;
a second step of placing a glass plate on the concave portions of the detection chip;
a third step of dropping a liquid which does not emit autofluorescence on the glass plate;
a fourth step of fixing the membrane filter on the glass plate;
a fifth step of placing the detection chip on the stage, the membrane filter being fixed on the detection chip;
a sixth step of emitting excitation light from the light emitter to the membrane filter;
a seventh step of taking an image of the membrane filter with the imaging unit; and
an eighth step of detecting the bacteria captured by the membrane filter on a basis of luminous points in the image taken by the imaging unit,
wherein, after fixing a membrane filter in one of the concave portions of the detection chip by performing processing of the first step to the fourth step using a fluorescent label capable of binding to both viable bacteria and dead bacteria, while the fluorescent label capable of binding to both viable bacteria and dead bacteria has been bound to bacteria captured by the membrane filter and
after fixing a membrane filter in another of the concave portions of the detection chip by performing processing of the first step to the fourth step using a fluorescent label capable of binding to viable bacteria or dead bacteria, while the fluorescent label capable of binding to either viable bacteria or dead bacteria has been bound to bacteria captured by the membrane filter,
the detection chip is placed on the stage by performing processing of the fifth step,
wherein a total number of bacteria is detected by performing processing of the sixth step to the eighth step on the membrane filter fixed on the one concave portion, and
wherein a number of viable bacteria or a number of dead bacteria is detected by performing processing of the sixth step to the eighth step on the membrane filter fixed on the another of the concave portions.
